(19) United States
(12) Patent Application Publication
(10) Pub. No.: US 2025/0161838 A1
(43) Pub. Date: May 22, 2025

(54) SEPARATION OF HYDROCARBONS BY EXTRACTIVE SOLVENT

(71) Applicant: Dev Mohan MOHUNTA, Chennai (IN)

(72) Inventors: Dev Mohan MOHUNTA, Chennai (IN); Karunakaran NAGARAJAN, Chennai (IN)

(21) Appl. No.: 18/730,293

(22) PCT Filed: Jan. 10, 2023
(86) PCT No.: PCT/IN2023/050022

§ 371 Date: Jul. 18, 2024

(30) Foreign Application Priority Data
Jan. 18, 2022 (IN) 202241002753

(51) Int. Cl. B01D 3/40; B01D 3/06; B01D 5/00; C07C 7/08

(52) U.S. Cl. CPC B01D 3/40; B01D 3/065; B01D 5/006; B01D 5/009; C07C 7/08

(57) ABSTRACT

The present invention discloses a process for extractive distillation for separating one or more hydrocarbons from a gaseous mixture. Briefly, the process disclosed in the present invention provide a process for separation of propylene from propane in simplified manner to maximize the propylene stream purity with very low concentration of propane. The process disclosed in the present invention is cost effective having significantly lower operating and capital cost and leads to maximizing the recovery and recycle of acrylonitrile and minimize the acrylonitrile as contaminant in propane stream.

### TECHNICAL FIELD OF THE INVENTION

The present disclosure generally relates to a process for separation of hydrocarbons, more specifically to C3 and C4 alkanes and alkenes. More particularly the disclosure relates to a process of separation of propane and propylene by the process of distillation in presence of acrylonitrile.

### BACKGROUND OF THE INVENTION

The separation of propane from propylene is an important industrial process, where the purity requirements of propylene may range from 98% to 99.5% depending upon the end use. The difference in boiling temperature between the two hydrocarbons is 5.5° C. making the separation process is difficult and expensive. The usual practice to do high pressure distillation (conventional) as shown in FIG. 1. The distillation pressure is usually dictated by gas composition and the cooling water temperature of the condenser. The number of trays may range from 150 to 250 trays. Thus, the column may sometimes have to be split into two where each column may be about 65 to 75 meters in height and a reflux ratio of 10-20. The equipment described above referred to as C3 splitter. Because of the high capital cost and operating cost numerous modifications have been practiced within the frame work of the aforementioned process to reduce utility costs. Other methods in combination with distillation and methods such as membrane separation, adsorption, etc. have also been described with the aim of reducing operating costs.

The researchers have been constantly working on the various processes of separation of propane and propylene. For instance, a patent document GB548733A discloses a separation of propane and propylene by extractive distillation and describes a test procedure for evaluating extractive solvents to be used in the extractive distillation method. It mentions and provides the relative efficacy of different solvents. The following were evaluated, furfural water, acetamide-water, acetonitrile, propionitrile and various mixtures Also, there is no mention of use of acrylonitrile.

In another non-patent document, Kumar R et al.; gives a theoretical methodology for comparison of extractive distillation solvents

Further, U.S. Pat. No. 5,085,741 shows that extractive distillation can be used for separation of propylene from propane by using propylene carbonate as an extractive solvent with or without small percentage of water.

U.S. Pat. No. 2,588,063 shows that acetonitrile alone or in combination with an amide can theoretically separate propane from propylene, propane will be in the gas phase. No details are mentioned. Bao liao et. al. has shown that acetonitrile-10% water mixture is a more effective solvent than indicated in U.S. Pat. No. 2,588,063. for extractive distillation of propylene-propane mixture. Relative volatility data at infinite dilution were calculated by UNIFAC method and experimentally verified, based on this design has been done. The extractive distillation column operates under a pressure of 1910 KPA so ordinary cooling water can be used. The feed is 84% Propylene 16% propane. The ratio of acetonitrile to feed is 2.33 moles However, the process used is complicated and energy intensive and requires dehydration of propylene.

The separation process envisages four sections. (1) extractive distillation column (2) Stripping column (3) water scrubber (4) column for acetonitrile recovery. In the extractive distillation column propane is obtained at the top of the column (FIG. 3). The bottoms consisting of propylene and acetonitrile mixture go to the stripping column, propylene of high concentration is obtained at top of the column and acetonitrile is recovered at the bottom. In the water column traces of acetonitrile are washed with water and pure propylene is recovered at top. Propylene is saturated with water vapour and requires to be dehydrated. The mixture of acetonitrile and water is distilled to recover acetonitrile. The process was simulated on commercial software PRO/II. A comparison is made with the existing process. WO2017085504A2 discloses use of large number of solvents for the separation of alkane-alkenes. However, the reference does not disclose the purity of propylene and separation of propane from other solvents. Further, the reference also does not disclose about the ratio of solvent to feed.

Among the large number of compounds listed for possible separation of propane-propylene the nitro compounds mentioned are acetonitrile, propionitrile, nitro methane, nitroethane, nitropropane and nitrobenzene but Acrylonitrile is not mentioned. The prior art only teaches measurement of equilibrium data and uses properties such as Hildebrand number, Hanson parameters, and polarity index to distinguish the efficacy of separation of propane-propylene among these compounds.

### OBJECT OF THE INVENTION

An object of the current invention is to provide a process for separation of propylene from propane in simplified manner.

Another object of the invention is to maximize the propylene stream purity with very low concentration of propane.

Another important object of the present invention is to achieve a much lower operating and capital cost.

Still another object of the present invention is to produce a propane stream with low concentration of propylene.

Yet another object of the present invention is to maximize the recovery and recycle of acrylonitrile and minimize the acrylonitrile as contaminant in propane stream.

### SUMMARY OF THE INVENTION

The principal embodiment of the present invention discloses a process for extractive distillation for separating one or more hydrocarbons from a gaseous mixture, the process comprising the steps of;
i. contacting the gaseous mixture of hydrocarbons with an extractant suitable for extracting the one or more hydrocarbons, wherein extractant uses acrylonitrile as the extractive solvent.
   wherein, propylene and propane mix with addition of acrylonitrile is separated in a distillation column operating at pressure of 1910 Kpa, the pressure in the distillation column may vary from 1600 to 2600 Kpa
   and wherein, the molar fraction of propylene-propane to Acrylonitrile is 0.7;
   molar fraction of extractant to hydrocarbon mix to obtain the desired separation ranges from 0.1 to 0.9
ii. condensing the propylene in gas phase at top is with cooling water;
iii. separating the mixture of propane and acrylonitrile in the liquid without reducing the pressure;
iv. heating the mixture is to predetermined temperature of 88° C. and flashing in a vessel where in the vapours go to a condenser to condense as acrylonitrile;
V. heating the liquid from the first flash vessel to higher predetermined temperature of 94° C. and flashing in second vessel;
vi. cooling the vapours from the second flash vessel in series of two condensers to recover the acrylonitrile wherein, the gases move to the hydrocarbon header;
vii. collecting the recovered acrylonitrile from the two flash vessels in collection tank and recycled to the contacting apparatus
viii. combining the propane vapours from the flash vessels in the header, wherein the pressure is reduced to 642 Kpa to go to a vessel for removal of last traces of acrylonitrile by water wash;
ix. separating and obtaining hydrocarbons in pure form.

Briefly, the process disclosed in the present invention provide a process for separation of propylene from propane in simplified manner to maximize the propylene stream purity with very low concentration of propane. The process disclosed in the present invention is cost effective having significantly lower operating and capital cost. The process disclosed in the present invention leads to maximizing the recovery and recycle of acrylonitrile and minimize the acrylonitrile as contaminant in propane stream.

### BRIEF DESCRIPTION OF THE DRAWINGS

FIG. 1 illustrates a conventional distillation process as per the state of the art.

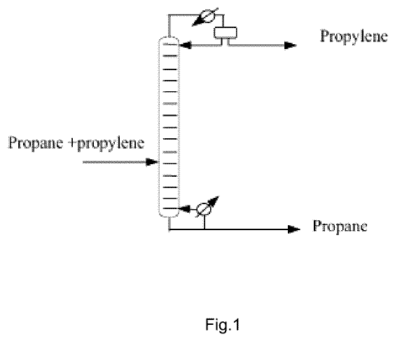

FIG. 2 illustrates an extractive distillation process of Propane and Propylene as per the state of the art.

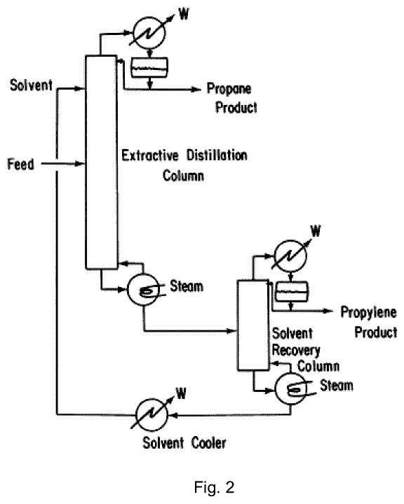

FIG. 3 illustrates prior art extractive distillation process using acetonitrile as extractive solvent

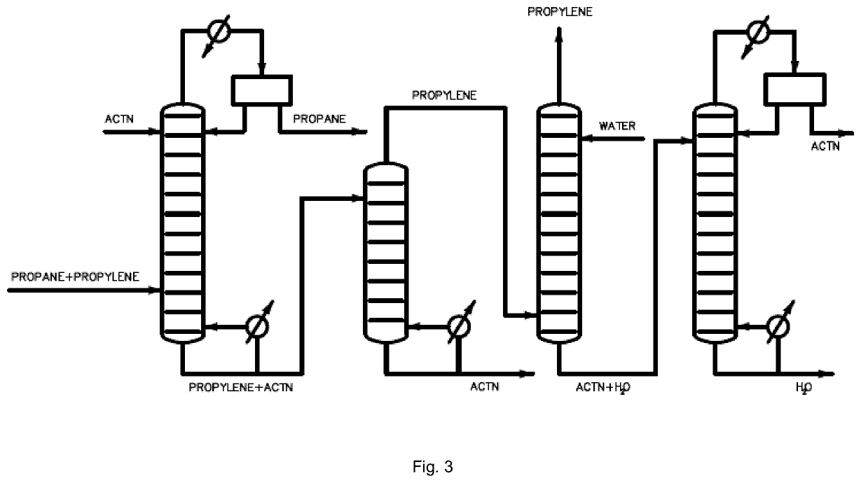

FIG. 4 illustrates the process of the present invention using acrylonitrile as extractive solvent.

### DETAILED DESCRIPTION OF THE INVENTION

The process as already described in general is extractive distillation to separate two hydrocarbons. However, the study on various extractive solvents leads to a fact that the volatility of propylene in acrylonitrile is very high as compared to propane. The higher the ratio, easier is the separation of hydrocarbons, where streams with high propylene concentration would separate out in first stage itself thus reducing the operating load on the remaining equipment that separate propane from acrylonitrile. In the prior art processes, propane, a lower percentage stream separates in the first stage, however the larger component propylene is processed in subsequent stages increasing the operating costs. Further in the conventional process a high reflux ratio of 10-20 has to be used, compared to reflux ratio of 2 in present embodiment.

Relative volatility is defined by the following equation where in present case a subscript A refers to propane and B refers to propylene. (Reid, Prausnitz)

$${a}_{A B} = \frac{( {γ}_{A} ) ( {P}_{A}^{0} )}{( {γ}_{B} ) ( {P}_{B}^{0} )}$$

Where P⁰ is the pure-component saturation (vapour) Pressure.
The above equation may be written as,

$${a}_{A B} = S ( \frac{{P}_{A}^{0}}{{P}_{B}^{0}} )$$

### Where

$$S = \frac{{γ}_{A}}{{γ}_{B}}$$

In an embodiment, the present invention discloses a process for extractive distillation for separating one or more hydrcoarbons from a gaseous mixture, the process comprising the steps of;
i. contacting the gaseous mixture of hydrocarbons with an extractant suitable for extracting the one or more hydrocarbons, wherein extractant uses acrylonitrile as the extractive solvent.
   wherein, propylene and propane mix with addition of acrylonitrile is separated in a distillation column operating at pressure of 1910 Kpa, the pressure in the distillation column may vary from 1600 to 2600 Kpa
   and wherein, the molar fraction of propylene-propane to Acrylonitrile is 0.7;
   molar fraction of extractant to hydrocarbon mix to obtain the desired separation ranges from 0.1 to 0.9
ii. condensing the propylene in gas phase at top is with cooling water;
iii. separating the mixture of propane and acrylonitrile in the liquid without reducing the pressure;
iv. heating the mixture is to predetermined temperature of 88° C. and flashing in a vessel where in the vapours go to a condenser to condense as acrylonitrile;
V. heating the liquid from the first flash vessel to higher predetermined temperature of 94° C. and flashing in second vessel;
vi. cooling the vapours from the second flash vessel in series of two condensers to recover the acrylonitrile wherein, the gases move to the hydrocarbon header;
vii. collecting the recovered acrylonitrile from the two flash vessels in collection tank and recycled to the contacting apparatus
viii. combining the propane vapours from the flash vessels in the header, wherein the pressure is reduced to 642 Kpa to go to a vessel for removal of last traces of acrylonitrile by water wash;
ix. separating and obtaining hydrocarbons in pure form.

In still another embodiment, said hydrocarbons are selected from, but not limited to alkanes and alkenes.

In yet another embodiment wherein said alkanes and alkenes are selected from, but not limited to propane and propylene.

In another embodiment the weight ratio of acrylonitrile to feed is 2.88:1.

In still another embodiment, the ratio of acrylonitrile to feed ranges from 0.1 to 0.9 mole fraction.

In yet another embodiment the ratio of acrylonitrile to feed ranges between 0.5 to 0.7 mole fraction.

In another embodiment the reflux ratio in the extractive distillation column is substantially lower than that for conventional high-pressure distillation.

The invention uses a novel extractive distillation solvent acrylonitrile that has not been used hitherto. The selection is proved by calculations that are common in such cases and well known in chemical engineering. Further its use results in significant economic benefits in terms of energy consumed. A further benefit of lower heat energy use is lower GHG emissions namely CO2 emissions, heat energy being derived from fossil fuels in such plants

### EXAMPLES

### Example 1

Table 1: comparative table showing the relative volatilities of propane to propylene for low concentration of the solute in solvent, 0.99:0.01 at 40° C. Calculated by UNIFAC method. The temp of 40° C. is chosen as it is close to the operating conditions.

TABLE 1

| | |
|---|---|
| Acetonitrile | 1.680992 |
| Propionitrile | 1.369684 |
| Butyronirile | 0.887504 |
| Acrylonitrile | 0.005676 |

The value for acetonitrile compared well with experimental and calculated values of 1.63 and 1.70 30° C. infinite dilution. (Bao liao et al.) It indicated that in case of acetonitrile and propionitrile, propylene was preferentially in the solvent and propane will be in the gas phase

The very low relative volatility for propane by acrylonitrile is important and indicates that propylene will be the overhead product or gas phase and propane and acrylonitrile will be the bottom product or liquid phase. A calculation of activity coeff. for the propylene-propane mixture for mole ratio of 2.77 moles acrylonitrile to 1 mole of mixture. Same Composition as indicated in Bo liao et. al.

TABLE 2
Alpha value for propylene

| | | | Vp mm | |
|---|---|---|---|---|
| Temp 40 C. | Mole frac. | gama | Hg | alpha |
| Acrylonitrile | 0.712 | 1.3973 | 201.66 | |
| Propylene | 0.229 | 7.1180 | 12147.8 | 306.87 |
| Propane | 0.059 | 0.9503 | 10141.7 | 34.20 |

The alpha value was high for propylene as shown. Activity coefficient of Propylene was much higher than that of propane, showing that there was a high concentration of propylene in the vapour phase compared to liquid phase. This is exactly reverse of that for acetonitrile. Therefore, for acetonitrile and nitriles other than Acrylonitrile the selectivity was high for propylene and it was in liquid phase and propane will be in vapour phase.

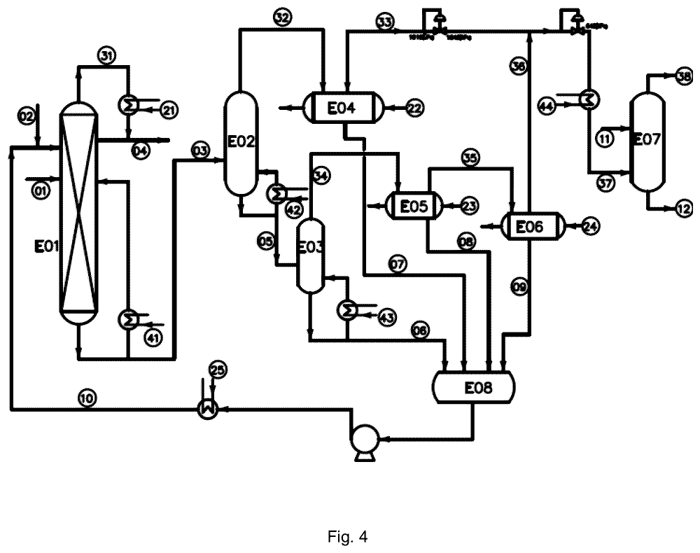

Simulation of the distillation column was done by open-source software DWSIM. Flow sheet is given in FIG. 4 The pressure in column, feed rate and feed composition were kept the same as in Bo Liao et. al. The pressure was 1910 KPa, temperature 45 C. the feed was liquid at this temperature. Table 3 illustrates the details of simulation. High purity propylene 99.3% mole purity is obtained without contamination with acrylonitrile. Manual calculations were also done using UNIFAC method.

TABLE 3

| | kg/hr | mass frac | mole frac |
|---|---|---|---|
| Feed | 125510 | | |
| Propylene | 26690 | 0.2127 | 0.252 |
| Propane | 5238 | 0.0424 | 0.048 |
| Acrylonitrile | 93491 | 0.7449 | 0.700 |
| Top temp | 46.4 | | |
| Product | 26083 | | |
| Propylene | 25892 | 0.9927 | 0.993 |
| Propane | 191 | 0.0073 | 0.007 |
| Acrylonitrile | 0 | 0 | 0 |
| bottom temp | 84.5 | | |
| bottoms | 99426 | | |
| Propylene | 798 | 0.0080 | 0.0100 |
| Propane | 5138 | 0.0518 | 0.0614 |
| Acrylonitrile | 93492 | 0.9402 | 0.9286 |

Many times the feed contains other contaminants like C2 ethane, ethylene, C4 butane etc. Because of the presence of butane the feed and operating pressures may be lower.

A typical feed composition is given below in Table 4

| | | |
|---|---|---|
| Press | 16.55 | atmos |
| Temp | 41.66 | C. |
| C2 | 0.0397% | Mole |
| Propylene | 72.81% | Mole |
| Propane | 26.48% | mole |
| C4 | 0.662% | mole |

A similar composition was taken for simulating extractive distillation the results are of feed and the product steams are given in Table 5. It will be noticed that the entire quantity of ethane goes with propylene at the top. Butane goes with the bottom stream containing propane. It has no effect on purity of propylene.

TABLE 5

| | | | Bottom | |
|---|---|---|---|---|
| | | Top | Propane + | |
| | Feed | Propylene | ACN | |
| Temperature | 45 | 46 | 84.5 | C. |
| Pressure | 1550 | 1550 | 1550 | kPa |
| Molar Flow | 2517.0 | 540.03 | 1976.97 | kmol/h |
| Volumetric | 189.53727 | 45.381165 | 158.19318 | m3/h |
| Flow | | | | |
| Ethane | 0.00012 | 0.00055 | 5.03E−47 | Mole frac |
| Propane | 0.07944 | 0.007 | 0.09922 | Mole frac |
| Butane | 0.00201 | 2.65E−11 | 0.002559 | Mole frac |
| Propylene | 0.21843 | 0.99243 | 0.007 | Mole frac |
| Acrylonitrile | 0.7 | 2.41E−24 | 0.89121 | Mole frac |

Referring to FIG. 4 there is shown a flow sheet, a bottom stream 03 from distillation column E01 at 1910 Kpa and 84.5 C that is flashed by increasing the temperature but maintaining the pressure as in the column. The stream 03 passes through series of heated flash columns E02 & E03. The first flash heater E02 operates at 88 C and a hot liquid stream 05 is fed to the second heated flash column E03 where it is heated to 94 C.

Vapours 32 from E02 is then sent to a partial condenser E04 and cooled to 51 C. The design of condenser efficiently separates the condensate from the vapour. A condensate 07 goes to a collection tank E08 and the vapours 32 go to a water wash column for removing acrylonitrile.

From E02, the liquid stream 05 from bottom is heated in E03 to 94 C. Vapours 34 are separated and a bottom liquid 06 from the second flash heater E03 at 94 C is cooled to 50 C and goes to a tank E08. The vapours 34 go to a partial condenser E05 and are cooled from 94 C to 80 C. A condensate 08 is cooled to 50 C and goes to the condensate Tank E08. This constitutes almost 99.9% of the acrylonitrile in circulation.

Vapours 35 from the partial condenser E05 go to a partial condenser E06 and are cooled to 55 C. The vapours 35 from this are combined with vapour from partial condenser E04 and are water washed to remove acrylonitrile. Thereafter, a condensate 09 goes to the tank E08.

The tank E08 is maintained at plant pressure and the collected acrylonitrile with some propane and propylene are recycled to the main distillation column.

The combined vapours from the partial condensers E04 and E06 are at high pressure and the pressure is let down through a valve from 1842 KPa to 642 Kpa. The temperature falls due to depressurization and are outlet stream from the pressure reduction valve is heated to 26 C wherein C3 stream is in vapour form. This stream is washed with water in a short column E07 to capture the acrylonitrile. The outgoing gas stream 38 is propane with 2.26% propylene saturated with water. The bottom stream 12 is a mixture of acrylonitrile and water that separates into 2 layers the top layer is acrylonitrile and the bottom layer is water that is discarded.

Details of all streams are given in Table 6,7 and 8.

TABLE 6

| | Process liquid streams | | | | | | | | | | | |
|---|---|---|---|---|---|---|---|---|---|---|---|---|
| | 01 | 02 | 03 | 04 | 05 | 06 | 07 | 08 | 09 | 10 | 11 | 12 |
| Pressure - KPa | 1910 | 1910 | 1910 | 1910 | 1910 | 1910 | 1910 | 1860 | 1842 | 1910 | 642 | 642 |
| Tmp. - deg. C. | 45 | 45 | 84.5 | 46.4 | 88 | 94 | 51 | 80 | 55 | 45 | 26 | 26 |
| Flow rate - | 32018 | 93492 | 99426 | 26083 | 98186 | 92788 | 1120 | 416 | 752 | 95076 | 540 | 607 |
| kg/hr | | | | | | | | | | | | |
| Propane | 5328 | — | 5136 | 191 | 4793 | 17 | 309 | 98 | 528 | 952 | — | — |
| Propylene | 26690 | — | 798 | 25892 | 16 | 0.03 | 697 | 0.20 | 2 | 700 | — | — |
| Acrylonitrile | — | 93492 | 93492 | — | 93377 | 92771 | 114 | 318 | 222 | 93424 | — | 67 |
| Water | | | | | | | | | | | 540 | 540 |

TABLE 7

| | Process gas streams | | | | | | | |
|---|---|---|---|---|---|---|---|---|
| | 31 | 32 | 33 | 34 | 35 | 36 | 37 | 38 |
| Pressure - Kpa | 1910 | 1910 | 1910 | 1910 | 1860 | 1842 | 642 | 642 |
| Temperature - | 46 | 88 | 51 | 94 | 80 | 55 | 26 | 26 |
| deg. C. | | | | | | | | |
| Mass flow | 78335 | 1240 | 120 | 5399 | 4983 | 4230 | 4350 | 4279 |
| rate - | | | | | | | | |
| kg/hr | | | | | | | | |
| Propane | 5725 | 343 | 34 | 4777 | 4678 | 4150 | 4184 | 4182 |
| Propylene | 72610 | 783 | 85 | 16 | 16 | 14 | 99 | 97 |
| Acrylonitrile | — | 114 | 1 | 606 | 289 | 66 | 67 | 0.06 |

TABLE 8

| | Utility streams | | | | | | | |
|---|---|---|---|---|---|---|---|---|
| | 21 | 22 | 23 | 24 | 41 | 42 | 43 | 44 |
| Pressure - KPa | | | | | 303.9 | 303.9 | 303.9 | 303.9 |
| Temperature - | 30 | 30 | 30 | 30 | 134 | 134 | 134 | 134 |
| deg. C. | | | | | | | | |
| Mass flow | 1077024 | 20502 | 16745 | 24486 | 14801 | 516 | 1115 | 154 |
| rate - | | | | | | | | |
| kg/hr | | | | | | | | |
| Steam | — | — | — | — | 14801 | 516 | 1115 | 154 |
| Water | 1077024 | 20502 | 16745 | 24486 | — | — | — | — |

The process is very advantageous as there is very small pressure change during the recovery and recycle of acrylonitrile thus avoiding recompression and large energy consumption. In the propane recovery section, the pressure reduction is minimized so that power for recompression is minimized.

The process is advantageous as the loss of acrylonitrile is less than 0.05% per hour of the circulating acrylonitrile.

It should be noted that circulating acrylonitrile is pumped with pressure differential of 68 Kpa plus the hydraulic head of column height which is 20-30% of conventional column. The total power consumption being much less than that for conventional distillation.

### Non-Restrictive Conditions

The process of extractive distillation with acrylonitrile can be used for separation of alkane and alkene in general and is not confined to Propane propylene. The foregoing calculations and details are for system acrylonitrile-propane-propylene and are not limited to the values mentioned. The ratio of extractive solvent to feed can be varied considerably. The ratio of extractant to feed may from 0.1 to 0.9 mole fraction preferably from 0.5 to 0.7 mole fraction. The methodology is equally valid for large range of concentrations, impurities, etc. pressure and other conditions that may vary from plant to plant. The pressure conditions are generally set by the consideration of composition of the feed stream and the temperature of cooling water.

The main objective of the part of the process that separates propane from acrylonitrile is to minimize the acrylonitrile content of propane and to maximize recycle of acrylonitrile. The example is illustrative only and considerable variations type of equipment and conditions are possible to achieve the stated goals. The pressure and temperatures are varied depending upon the composition of the stream.

The savings in energy consumption is indicated. It is also possible to further reduce energy consumption by exchange of high and low energy levels and various other techniques known to those familiar with art.

### Energy Consumption

In the decision for adoption of technology energy consumption is key criteria. Energy means heat energy as well energy to drive rotary equipment. Comparative operating costs are required for making a decision. Table 6 gives the comparative energy consumption at two levels for the conventional process, acetonitrile process and the invention.

TABLE 9

| | | | Conventional | Acetonitrile + | |
|---|---|---|---|---|---|
| S. No | Heat load | UOM | distillation | Water | Acrylonitrile |
| | Distillation column | | | | |
| 01 | Condenser | Kcal/hr | 29205800 | 2342200 | 5385123 |
| 02 | Reboiler | Kcal/hr | 29158000 | 11711000 | 7644846 |
| | Extractive | | | | |
| | distillation stage | | | | |
| 01 | Heating - 1 | Kcal/hr | NA | 8938600 | 266628 |
| 02 | Heating - 2 | Kcal/hr | NA | 4660500 | 575868 |
| | Total | Kcal/hr | NA | 13599100 | 842496 |
| | Solvent Recovery | | | | |
| | stage | | | | |
| 01 | Condensing - 1 | Kcal/hr | NA | NA | 83724 |
| 02 | Condensing - 2 | Kcal/hr | NA | NA | 102513 |
| 03 | Condensing - 3 | Kcal/hr | NA | 3776200 | 122431 |
| | Total | Kcal/hr | NA | 3776200 | 308668 |
| | Total Energy | | | | |
| 01 | Heat Energy | Kcal/hr | 29158000 | 25310100 | 8487341 |
| 02 | Cooling Energy | Kcal/hr | 29205800 | 6118400 | 5693791 |
| | Energy Saving | % | | 13.19 | 70.89 |
| | (Heating) | | | | |
| | Energy Saving | % | | 79.05 | 80.50 |
| | (Cooling) | | | | |

The invention saves 70.89% of heat energy compared to conventional process, and 80.50% energy for cooling water. Compared to acetonitrile (Ba Liao), the present invention saves 66.5% heat energy and 7% of cooling load.

### Improved Column Configuration

In conventional Propane-Propylene distillation columns the actual numbers of trays may range from 150 to 250 trays. Instead of one there could be two columns. A large variety of trays have been employed based on claims of efficiency, pressure drop etc. When extractive distillation is practiced as stated in this invention the number of trays are drastically reduced. In the illustration above the number of trays is about 30. The number of trays would vary depending upon the mole fraction of acrylonitrile in feed. If the mole fraction is 0.1 of feed the number of trays is 100, if the mole fraction is 0.85 the number of trays is 7. For the preferable range of feed compositions, it would be possible to use packed columns. A large variety of packings are available and can used. There will be significant reduction in column height and simplifying column fabrication resulting in lower capital cost.

### Improved Life of Acrylonitrile

The use of Acrylonitrile may encounter difficulties as it is known to self-polymerize by a free radical mechanism. Polymerization affects the liquid properties as well as cause blockage of equipment. This can be inhibited by the addition of inhibitor like a quinone, such as TBHQ. Thus for long life of acrylonitrile and trouble free operation of equipment constant monitoring and injection of TBHQ is required.

The foregoing descriptions of specific embodiments of the present disclosure have been presented for purposes of illustration and description. They are not intended to be exhaustive or to limit the present disclosure to the precise forms disclosed, and obviously many modifications and variations are possible in light of the above teaching. The exemplary embodiment was chosen and described in order to best explain the principles of the present disclosure and its practical application, to thereby enable others skilled in the art to best utilize the present disclosure and various embodiments with various modifications as are suited to the particular use contemplated.

### Advantages of the Invention

The foregoing description indicates that the aim of separation and a high purity of propylene is achieved, and the capital cost is substantially lower than the existing solutions, as:
1. The number of theoretical plates is about 30 that is 15% compared to conventional distillation process or variants thereof.
2. The column's internal structure can be packings and does not need trays as in conventional columns.
3. An extractive distillation process that can be operated at different pressures as required by the upstream process and composition of feed.
4. There is considerable decrease in the energy consumption. The process decreases energy use be as much as 70% to 80% over conventional high-pressure distillation
5. As. compared to other extractive distillation systems separation of acrylonitrile-propane does not require a number of distillation columns.
6. The process is equally valid for large range of concentrations, impurities, etc. pressures
7. A process advantageously flashes by temperature increase the recovery of propane at high pressure in single or multiple stages.
8. The recovered acrylonitrile is also under pressure so that cost and energy of pumping back to the column is reduced.
9. The process of separation may also be achieved by decreasing the pressure in stages and recovering the streams.

What is claimed:

1. A process for extractive distillation for separating one or more hydrcoarbons from a gaseous mixture, the process comprising the steps of;
i. contacting the gaseous mixture of hydrocarbons with an extractant suitable for extracting the one or more hydrocarbons, wherein extractant uses acrylonitrile as the extractive solvent,
wherein, propylene and propane mix with addition of acrylonitrile is separated in a distillation column operating at pressure of 1910 Kpa, the pressure in the distillation column may vary from 1600 to 2600 Kpa
and wherein, the molar fraction of propylene-propane to Acrylonitrile is 0.7;
molar fraction of extractant to hydrocarbon mix to obtain the desired separation ranges from 0.1 to 0.9
ii. condensing the propylene in gas phase at top is with cooling water;
iii. separating the mixture of propane and acrylonitrile in the liquid without reducing the pressure;
iv. heating the mixture is to predetermined temperature of 88° C. and flashing in a vessel where in the vapours go to a condenser to condense as acrylonitrile;
V. heating the liquid from the first flash vessel to higher predetermined temperature of 94° C. and flashing in second vessel;
vi. cooling the vapours from the second flash vessel in series of two condensers to recover the acrylonitrile wherein, the gases move to the hydrocarbon header;
vii. collecting the recovered acrylonitrile from the two flash vessels in collection tank and recycled to the contacting apparatus
viii. combining the propane vapours from the flash vessels in the header, wherein the pressure is reduced to 642 Kpa to go to a vessel for removal of last traces of acrylonitrile by water wash;
ix. separating and obtaining hydrocarbons in pure form.

2. The process as claimed in claim 1, wherein said hydrocarbons are selected from, but not limited to alkanes and alkenes.

3. The process as claimed in claim 2, wherein said alkanes and alkenes are selected from, but not limited to propane and propylene.

4. The process as claimed in claim 1, wherein the weight ratio of acrylonitrile to feed is 2.88:1.

5. The process as claimed in claim 1, wherein the ratio of acrylonitrile to feed ranges from 0.1 to 0.9 mole fraction.

6. The process as claimed in claim 1, wherein the ratio of acrylonitrile to feed ranges between 0.5 to 0.7 mole fraction.

7. The process as claimed in claim 1, wherein the reflux ratio in the extractive distillation column is substantially lower than that for conventional high-pressure distillation.
